(19) United States
(12) Patent Application Publication
(10) Pub. No.: US 2015/0140634 A1
(43) Pub. Date: May 21, 2015

(54) METHOD FOR PRODUCING A POLYMER PRODUCT FROM MULTIDIMENSIONAL AGGREGATED COMPONENTS AS BARRIER OR CARRIERS OF LIVING MICROBIAL CELLS AND BIOLOGICAL BARRIERS IN PLASTIC AND TEXTILE

(71) Applicant: RESILUX N.V., Wetteren (BE)

(72) Inventors: William DIERICKX, Destelbergen (BE); Dirk DE CUYPER, Destelbergen (BE); Nina GUNDE-CIMERMAN, Ljubljana (SI); Natasa POKLAR ULRIH, Ljubljana (SI)

(21) Appl. No.: 14/309,760

(22) Filed: Jun. 19, 2014

Related U.S. Application Data
(63) Continuation of application No. 14/066,535, filed on Oct. 29, 2013, now abandoned.
Application No. 14/066,535 is a continuation of application No. PCT/BE2012/000022, filed on May 2, 2012.

(30) Foreign Application Priority Data
May 18, 2011 (BE) 2011/0303

(51) Int. Cl. C12N 11/04; B29D 22/00; B29C 49/00; B29C 37/00

(52) U.S. Cl. CPC C12N 11/04; B29C 37/0025; B29D 22/003; B29C 49/0005; B29K 2067/003
USPC 435/182; 264/112; 264/537

(57) ABSTRACT

This invention relates to a polymer product comprising the components consisting of a polymer in form of granules; a multidimensional structure containing amphiphilic molecules, such as lipids, surfactants or polymers, wherein said polymer product has an active and/or passive oxygen or CO₂ barrier, with an UV blocking activity or with potentially colouring with the incorporation of the multidimensional structure. It further relates to a method for manufacturing notably said polymer products, in particular from multidimensional aggregated components as barrier or carriers of living microbial cells and biological barriers in plastic and textile.

### FIELD OF THE INVENTION

This invention relates to components that, within a matrix, have the ability to aggregate to multidimensional structures, and to organisms containing such components and their use as solute, gas, thermal and UV-barriers in plastic materials and textile, and to thus formed multidimensional structures as carriers of living microbial cells and their use as solute, gas, thermal and UV-barriers in plastic materials and textile.

### PRIOR ART

Several components can aggregate within a matrix, and form multidimensional structures. This matrix can be an aqueous or a non-aqueous liquid or a polymeric solid material. These multidimensional structures can vary from being two-dimensional, e.g. planar, to more complex three-dimensional structures.

Not only polymers can aggregate in a matrix to form multidimensional structures as disclosed by Holmberg et al. chapter 12, but also smaller amphiphilic molecules can do so as taught by Holmber et al., chapter (Evans and Wennerstrom, chapter 1).

Examples of multidimensional structures are vesicles consisting of one or more closed bilayer shells made of amphiphilic molecules, surrounding an aqueous core. These amphiphilic molecules comprise a hydrophobic and a hydrophilic part. These amphiphilic molecules can be surfactants Typical for vesicle forming surfactants is that the projected surface area of the hydrophobic group is about equal to the projected surface of area of the hydrophilic group. Vescile-forming surfactants are natural, such as phospholipids, or synthetic, such as double-tailed quaternary ammonium halides. These surfactants can be negatively charged, such as the phospholipid phosphatidyl glycerol, zwitterionic, such as the most abundant phospholipid phosphatidyl choline or positively charged, such as the double-tailed dialkyldimethalammonium halides (Cocquyt, 2005). Petkova et al. (2003) and Platikanov (2003) have investigated the gas permeability of phospholipid bilayers using a very specific bubble diminishing method.

In another example, the polar head groups of archaeal lipids are phospholipids or glycosides that are linked to one of the core lipids. The most common phospholipids are phosphoserin, phosphoinositol, phosphoglycerol, phosphoetanolamin and many phosphoglyicolipids, among them the most common carbohydrates found among archaeal lipids are glucose, gulose, mannose, galactose, inositol and N-acetylglucosamine, which can form mono-, di-, or oligosaccharides on one or both sides of caldarchaeol. Phosphoglycolipids with two polar head groups on both sides of the caldarchaeol may have glycerophosphate as the phosphoester moiety on one side and gulose alone or glucose and mannose, which form mono-, di-, or oligosaharides as the sugar moiety on the other side as is in the case of Thermoplasma acidophilum (Shimada et al. 2008). Replacement of one glycerol moiety of the core lipid backbone by a nonitol has also been observed (De Rosa and Gambacorta, 1988). The occurrence of unusual carbohydrate β-D-galactofuranosyl units has been found in methanogens (Gambacorta et al., 1995), but it has not been found in thermoacidophiles (Tables 1 and 2) since the five-member rings in such environments are rapidly hydrolyzed.

For instance, with regard to Barrier for O₂, CO₂, H⁺ protons, small organic molecule, UV-light and heat, ether linkages are more stable than ester over a wide range of pH, and the branching methyl groups help to reduce both crystallization (membrane lipids in the liquid crystalline phase at ambient temperature) and membrane permeability (steric hindrance of the methyl side group). Ether lipids are also resistant to enzymatic degradation by phospholipases and archaeal liposomes are exceptionally stable, they do not fuse or aggregate during storage at 4° C. over a period of 4 months (De Rosa, 1996). The saturated alkyl chains would impair stability towards oxidative degradation particularly in halophiles that are exposed to air and sunlight (Benvegnu et al., 2008). The membranes of methanogens and thermoacidophiles essentially consist of bipolar monolayer structures (Table 2). The high proportions of glycosylated lipids presented in membranes of thermoacidophiles and methanogenes may further stabilize their membrane structures through the interglycosyl headgroup hydrogen bonding. The presence of large sugar heads towards the convex surface of the membrane is likely to promote an asymmetric orientation, thus making the monolayer organization easier. Furthermore, the flux of small molecules and protons through archaeal bipolar tetraether lipid membranes is considerably reduced as the result of the particular physical structure of the lipid monolayer. Finally, the presence of the cyclic diether structures in species isolated from deep-sea hydrothermal vents may be related to the high pressures under which these archaea live.

It is known that the structural differences between lipids affect solute permeation through the membranes. In the polar head group regions of bipolar tetraether liposomes, there is an extensive network of hydrogen bonds, which should generate a high electrical dipole potential, thus hindering solute permeability through membranes.

For example, as to artificial lipid membranes, in particular planar lipids, more particularly liposomes, lipids were shown to be an excellent source for the formation of liposomes with thermostability and tightness against solute leakage (Gambacorta et al. 1995).

Membrane stability can be conveniently monitored by determining the release of fluorescent dyes originally trapped in the intravesicular compartment of the liposomes. In particular, several studies have focused on investigation of membranes made solely from bipolar lipid fractions. Different physicochemical properties, such as structure, dynamics, and polymorphism, thermal and mechanical stability, of bipolar lipid fractions extracted from several archaeal species have been investigated (Chang 1994; Elferink et al. 1994; Yamauchi et al. 1993).

Arakawa and his coworkers (Arakawa et al. 1999) investigated the polymorphism and physicochemical properties of the macrocyclic lipids by synthetic 72-membered macrocyclic tetraether lipids. DSC, ³¹P NMR and electron microscopy analysis studied the physicochemical features of diphospholipids. The cyclic tetraetherlipids appeared to show lower phase transition temperature (T_{c}). Fluorescence studies have shown that the passive proton permeability in bipolar tetraether liposomes isolated from S. acidocaldarius is lower and less temperature sensitive than that in liposomes composed of monopolar diester lipids, although the permeability increases with temperature in all liposomes. It has been proposed that low proton permeability is due to the chemical structure of tetraether lipids and their monolayer organization, especially the cyclopentane rings and the network of hydrogen bonds between the sugar residues exposed at the outer face of the membrane (Chong et al. 2003, Mathai et al. 2001).

In general, it was shown that at a given temperature, the bolaform lipid chains are more ordered and less flexible than in conventional bilayer membranes. Only at elevated temperatures (80° C.) does the flexibility of the chain environment in tetraether lipid assemblies approach that of fluid bilayer membranes (Bartucci et al. 2005). By examination of water, solute (urea and glycerol), proton and ammonia permeability of archaeosome, it was shown that macrocyclic archaeol and caldarchaeol lipids reduced the water, ammonia, urea and glycerol permeability significantly (6-120-fold) compared to dipalmitoylphosphatidylcholine (DPPC) liposomes (Mathai et al. 2001).

### OBJECT OF THE INVENTION

The invention aims at proposing a polymer product providing a contribution over the known materials and applications considering the drawbacks and shortcomings thereof.

### SUMMARY OF THE INVENTION

For this purpose, there is presented a polymer product according to the invention as defined in the appended main claim. The invention leads to a polymer product containing beside the polymer also a multidimensional structure. The multidimensional structure can be synthetic, it can be derived from a cell membranes of a living organism or it can be a living organism. Further the multidimensional structure can also serve as carrier for a living organism in this polymer product.

The first aspect of the invention comprises the selection of amphiphilic molecules that have the ability to aggregate to multidimensional structures in an aqueous or a non-aqueous liquid or a polymer matrix. These amphiphilic molecules can be lipids found in cell membranes of living organisms, but they can also be synthetic compounds like surfactants or polymers. The multidimensional structure is typically a microemulsion, an L3 (sponge) phase, a hexagonal or a lamellar structure such as a vesicle or liposome,

In a second aspect of the invention, the multidimensional structure can also be a living organism. The structure of the cell membrane of living organisms is very similar to the structure of a unilamellar vesicle.

In a third aspect of the invention, the multidimensional structure is composed of archaeal lipids.

In a fourth aspect of the invention, the multidimensional structure composed of amphiphilic molecules is used as a carrier for living organisms. This bioaggregate structure is then incorporated in the polymer product.

In all mentioned aspects of the invention, due to the incorporation in the polymer product of the multidimensional structure containing or not containing microbial cells or spores, a polymer product with improved properties is obtained compared to the polymer product where the multidimensional structure, containing or not containing the microbial cells, was not incorporated.

With regard to the physicochemical properties of components that, within a matrix, have the ability to aggregate to multidimensional structures, their potential applications are not yet disclosed. As part of the invention, the abovementioned structures, with or without included microbial cells or spores, can be used in the production of plastic materials and textile to achieve considering the following:
Lower permeability for hydrogen protons. In textile industry this means protections against acidic environment (lab coat, glows, . . . )
Lower and controlled permeability for O₂, CO₂ (in production of plastic bottles for soft drinks, fruits juice, olive and other oil sensitive to oxidation processes, . . . )
Lower permeability for smaller organic molecule (water filters for removing organic components
Lower permeability for water (water proofed textile, . . . )
Protection against heat and UV-light (textile, plastic, . . .
Coloring with biological pigments, included in the cell wall or membrane of the microbial cells. Additionally coloring prevents oxidation of products in the bottle, due to scavenging abilities of pigments.

According to a particular embodiment of the fourth aspect of the invention, living organisms can be selected from a category known as prokaryotic or eukaryotic vegetative cells and/or a phase of inactive or dormant stage such as sexual or asexual spores or cysts or meristematic clumps.

According to a more particular embodiment of the invention, said cells, mersistematic clumps or cell dormant stages, encapsulated in liposomes should withstand extremely dry conditions and temperatures well above 100° C. and act as permanent oxygen or CO₂ barriers, UV blockers and potentially colorings.

According to an advantageous embodiment of the invention, a reliable, slow and prolonged diffusion of organic molecules can be achieved through said polymers into said liposomes and to the encapsulated microbial cells, being realized, creating in the polymer a moist and fluctuating environment, that could activate slow metabolic microbial processes.

Depending on the selected microbial cell type, its metabolic activity within the liposome included in the polymer, the composition of the selected liposome as carrier, the selected liposome could or could not be degraded, enabling enlargement of immediate space within the polymer for the selected encapsulated microbial cell, enabling its slight expansion or growth and basic nutrition, creating an active and/or passive barrier for oxygen ad other gases permeation.

According to a further embodiment of the method of the invention, the bio component of the liposome—bioagreggates could be a type of yeast with a dry spore, such as for example Saccharomyces cerevisiae, which would be able to withstand the physicochemical conditions required for the inclusion of liposome—bio-aggregates into the polymer.

Another type of yeast could be pleomorphic yeasts such as Hortaea werneckii, able to grow as yeast cells, hyphal cells, budding hyphae, spores or meristematic clumps. Another type of fungal cells are molds such as Aspergillus spp. and their conidia, sexual spores, hyphal fragments, mycelial strands and dormant structures such as chlamidial cells or sclerotia.

Instead of fungal cells, algal cells could be incorporated into the liposome—bioagreggates, such as Haematococcus or Dunaliella salina, or their spores.

The bio component could be also a mixture of algal and fungal cells.

The bio component of the liposome—bioagreggates could be prokaryotic cells such as a type of bacterial cell with a dry spore or endospore, like for example Bacillus spp. or lactic acid bacteria or an archeal cell such as halophilic Halococcus or thermophilic Aeropyrum pernix.

According to a specific embodiment of the method according to the invention, said living organisms are selected from among the extremophiles.

According to a more specific embodiment of the invention, said living organisms are selected from among the species Bacillus Subtilis, in particular the one bearing No. ID9698.

According to a still more specific embodiment of the invention, said polymers are selected from among the family of the thermoplastic polymers, in particular from among the family of the polyolefins, or polyesters, more particularly from among the family of the polyethylenes, or polypropylenes, even more particularly polyethylene terephthalate (PET), yet more particularly in the form of PET granules that are coated with living organisms brought to a temperature higher than 260° C. during the injection molding process.

According to an alternative embodiment of the invention, said polymer is made of PETG, with a lower melting temperature than standard PET.

According to a useful embodiment of the invention, said product is a preform intended to be further processed to a container, in particular wherein said preform consists in at least three layers, an intermediate layer made of PETG between two outer layers, more particularly wherein said outer layers are made of PET, even more particularly wherein the injection molding is a co-injection molding where the PETG is injected colder than the layers on both sides of it, even more particularly wherein the PETG granules are coated with living organisms, and in wherein it is brought to a temperature higher than 200° C. during the injection molding process.

According to a further alternative embodiment of the invention, said product is a polymer film.

According to an additional embodiment of the invention, said living organisms are introduced in the polymer in a liquid stage during injection molding, at a temperature above 260° C.

According to a further additional embodiment of the preform according to the invention, it is intended for producing containers made of a polymer containing living organisms, consisting of at least one layer, wherein said layer is made of a polyethylene terephthalate glycol containing living organisms, in particular wherein said preform consists in at least three layers, the layer made of PETG being an intermediate layer between the two other layers, more particularly wherein said polymer product comprises a layer of a preform intended to make a container, said layer being a barrier layer, preferably a gas barrier layer, more preferably an oxygen barrier layer, more particularly wherein said living organisms in the PETG belong to the species Bacillus Subtilis, yet more particularly wherein said living organisms are introduced in the PETG in a liquid stage during injection moulding, at a temperature above 200° C.

According to a still further additional embodiment of the invention, a tertiary material is introduced in the middle layer material.

According to an advantageous embodiment of the invention, said tertiary material consists of a fluid. Thanks to the use of a fluid as tertiary barrier material, the barrier layer can be included with a specific additional function that is originating from said tertiary material with improved mechanic and/or thermal and/of barrier properties.

According to an additional advantageous embodiment of the invention, said fluid is a determined liquid. The fluid material has its liquid phase at least during, possibly after the production process, wherein said secondary layer is a barrier layer. Each layer is substantially continuous and uninterrupted. A tertiary material is thus incorporated therein as a fluid that helps to the creation of the barrier layer. This offers the advantage that it forms an efficient barrier along the preform—and thus the container as well—possibly thereby including its neck part. In this respect, the best tertiary materials are those having a lower viscosity under normal working conditions of temperature and pressure, referred to hereafter as cold liquids

According to a still further embodiment of the invention, said polymer contains at least two different types of living organisms.

Finally, the present invention also relates to a container made from a preform as defined in any of the appended sub-claims directed thereto, in particular wherein said living organisms in said container are active in a temperature range reaching at least 4 to 30° C. once the container has been filled, more particularly wherein at least two different types of living organisms are selected for coating granules.

Further features and properties are defined in the appended sub-claims.

Further details and particularities are illustrated in the following non limiting examples with reference to the attached drawings, which are described below.

### BRIEF DESCRIPTION OF THE DRAWINGS

FIG. 1 shows, as an example, typical structures of archaeal phospholipid compared with bacterial phospholipid.

FIGS. 2A-2L show various examples of different amphiphilic molecules.

FIG. 3 shows typical examples of multidimensional structures formed by amphiphilic molecules.

FIG. 4 is a diagrammatic representation of a cross section in a central plane of an embodiment of a preform according to the invention.

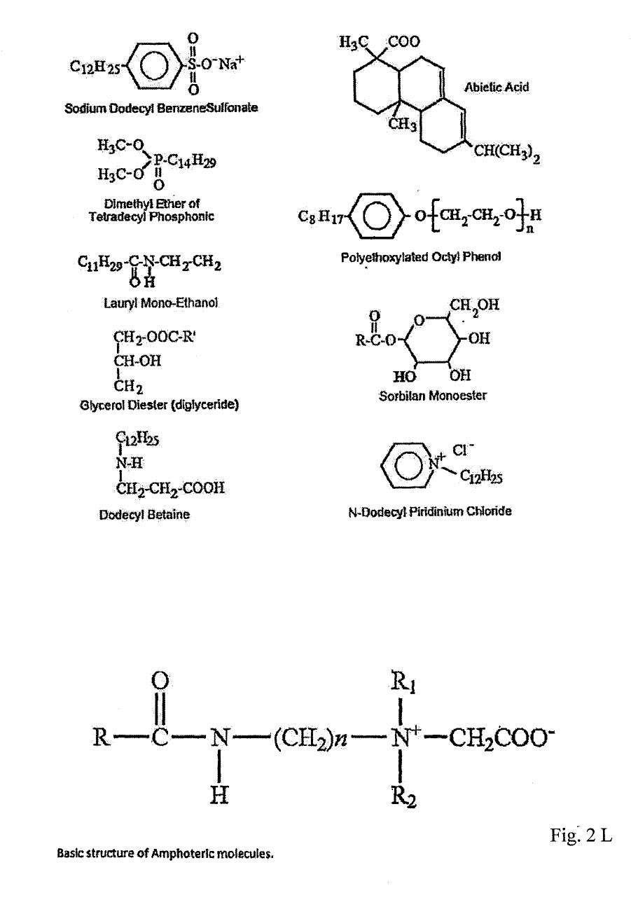

FIG. 5 is a mixed representation of a container according to the invention partially in a front view and partially in a cross section.

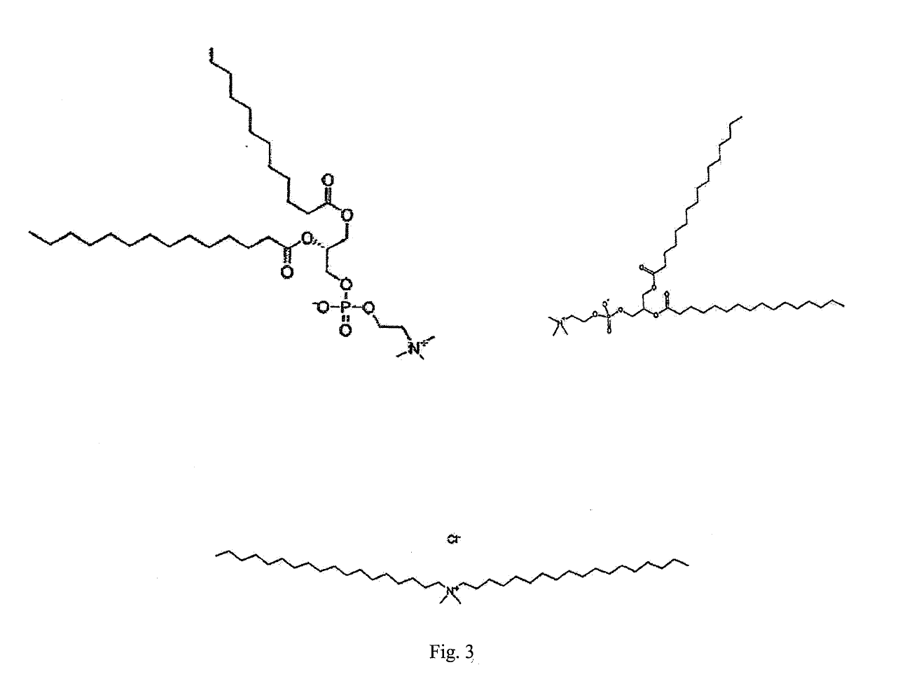

FIG. 6 is an enlarged view of a detail of the wall of the container represented in FIG. 4 according to the invention.

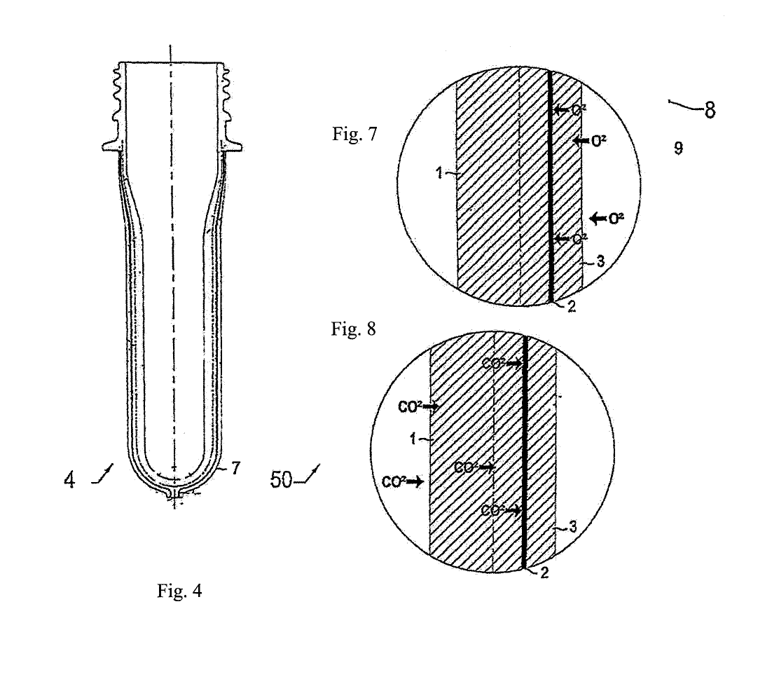

FIGS. 7 & 8 are each a diagrammatic representation of the functional working of an essential part of said container according to the invention, with an enlarged view of a detail of the wall thereof.

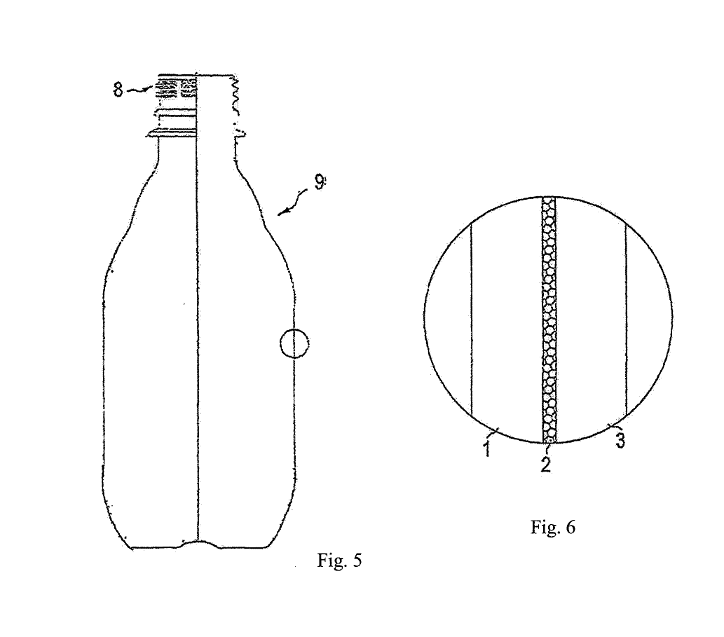

FIGS. 9 to 12 are an enlarged view of a detail of the perform resp container according to the invention

### DESCRIPTION

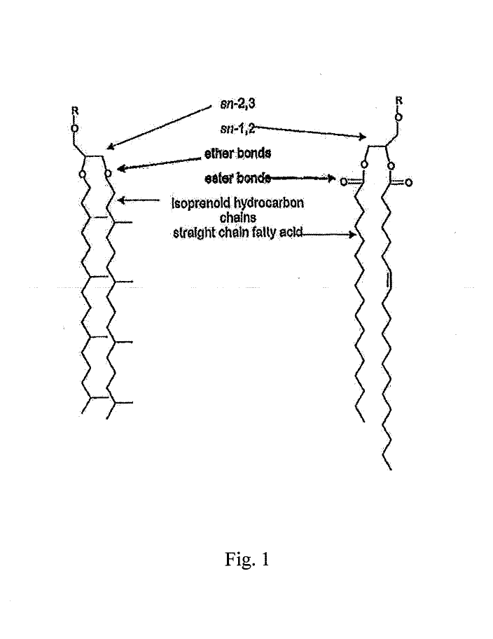

FIG. 1 shows, as an example, typical structures of archaeal phospholipid compared with bacterial phospholipid, wherein four differences are shown, −R standing for polar head group.

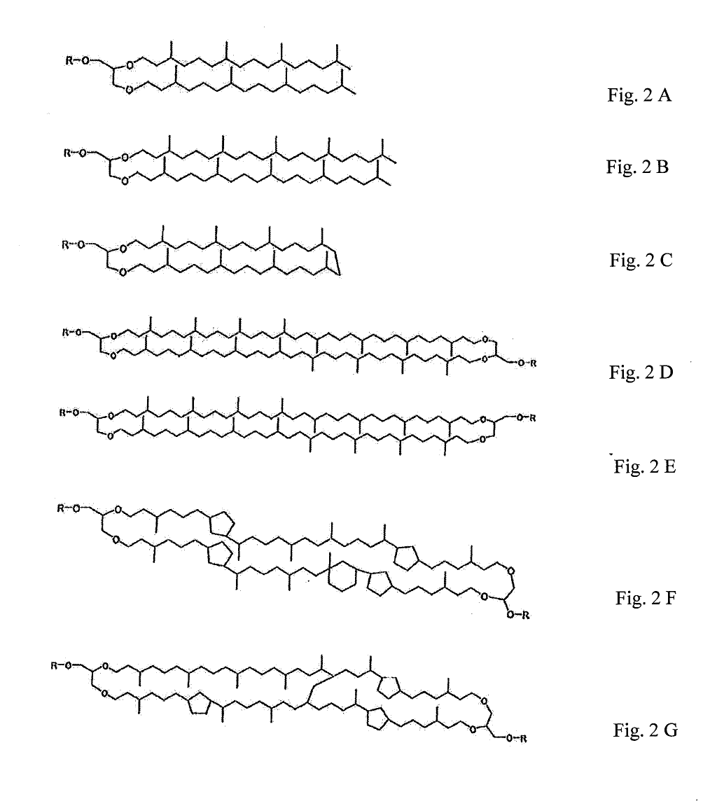

FIG. 2 shows some examples of different amphiphilic molecules, wherein both synthetic and natural molecules are shown. (a) archaeol (C20,20), (b) archaeol (C25,25), (c) macrocyclic arhaeol, (d) caldarchaeol, (e) isocaldarchaeol, (f) crenarchaeol, (g) H-shaped caldarchaeol containing three cyclopentane rings, (h). Sodium dodecylbenzene sulfonate, (i) Sodium stearate, (j) Trimethylhexadecyl ammonium chloride, (k) polyethylene ethoxylate, (l) basic structure of amphoteric molecules.

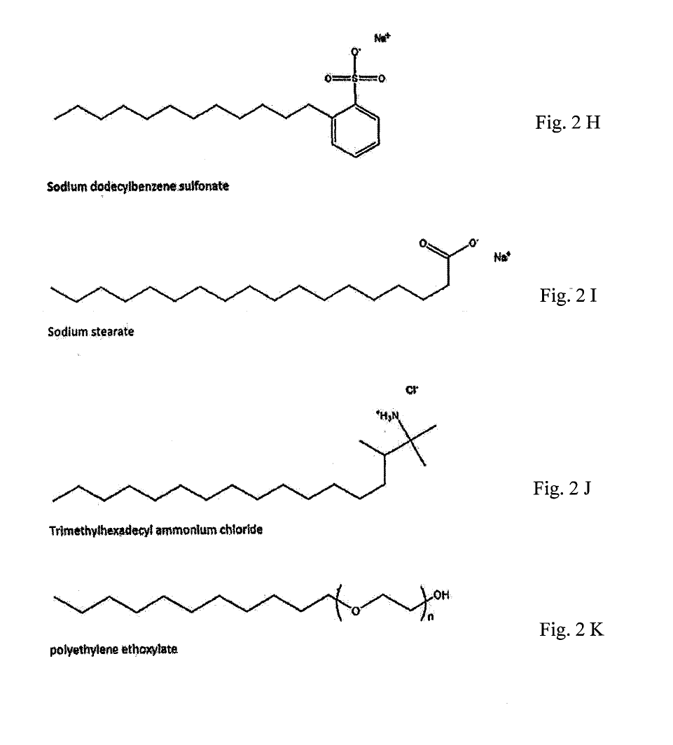

FIG. 3 shows typical examples of multidimensional structures formed by amphiphilic molecules including (a) spherical micelles, (b) cylindrical micelles, (c) planar bilayers, (d) inverted micelles, (e) bicontinuous structures, (f) bilayer vesicles.

The present invention generally relates to a polymer product, that comprises the following components:
a polymer in form of granules;
a multidimensional structure containing amphiphilic molecules, such as lipids, surfactants or polymers, wherein
the incorporation of the multidimensional structure leads to said polymer product with an improved active and/or passive oxygen or CO₂ barrier, with an improved UV blocking activity or with potentially colouring.

Its multidimensional structure is a micro-emulsion, an L3 (sponge) phase, a hexagonal or a lamellar structure such as vesicles or liposomes. It contains synthetic amphiphilic molecules such as surfactants or polymers. However, it may contain amphiphilic molecules commonly found in cell membranes of living organisms as well. It may also contain amphiphilic molecules that are found in archaeal micro-organisms.

Said multidimensional structure is used as a carrier for living microorganisms or spores. It is actually a living organism.

In a particular embodiment, said polymer product consists of a hollow form, more particularly of a preform for manufacturing containers 50, that is composed of a multilayer structure comprising at least a first double base layer, that is composed of a primary material A consisting of a polymer, thereby forming a double primary base layer 11 as embeddings matrix, that forms the outer surface of the preform that delimitate same, and that is further comprised of a secondary intermediate layer 12 which is comprised in said double primary basis layer 11, comprising a secondary material B, wherein said secondary material B consists of a support material wherein a tertiary material C is incorporated as initiator or resp. activator material thereby forming an inner composed barrier intermediate layer 12.

Said preform comprises a neck section 8, an adjoining wall section 6 and a bottom section 7 which forms the base of the preform, which is composed of a multilayer structure comprising at least three layers, the one of which 1 is directed inwardly respective the preform and is composed of a primary material thereby forming a primary basis layer 1, wherein said primary material consists of a synthetic material, and wherein a further layer 3 is directed outwardly with respect to said primary layer 1, in such a way that it forms the outer surface layer 3 of the preform, thereby consisting of a tertiary material forming a tertiary surface layer 3, wherein said tertiary layer is composed of a further synthetic material, characterized in that there is provided an intermediate layer 2 between the primary and tertiary layers 1, 3 which is composed of a secondary material consisting of a fluid.

Said primary material A consists of a plastic polymer, whereas said carrier material B consists of a polymer, in particular a plastic polymer, possibly a biopolymer, and said tertiary activator material C consists of a fluid. Said fluid is formed by a liquid, in particular a viscous liquid, more particularly an oil containing liquid, a water containing or water linked liquid, or an acrylate, possibly on oil or water basis.

Said secondary layer 12 may consist of a so-called intermediate phase such as tastes, adhesives and further materials having a liquid phase under normal conditions of pressure and temperature, and which may possibly get transformed into a solid phase, in particular by hardening.

Said preform is intended for being processed to containers, thereby possibly advantageously comprising a polymer containing living organisms, consisting of at least one layer 2, that is made of a polyethylene terephthalate glycol containing living organisms. Said living organisms are introduced in the polymer in a liquid stage during injection molding, e.g. at a temperature above 260° C.

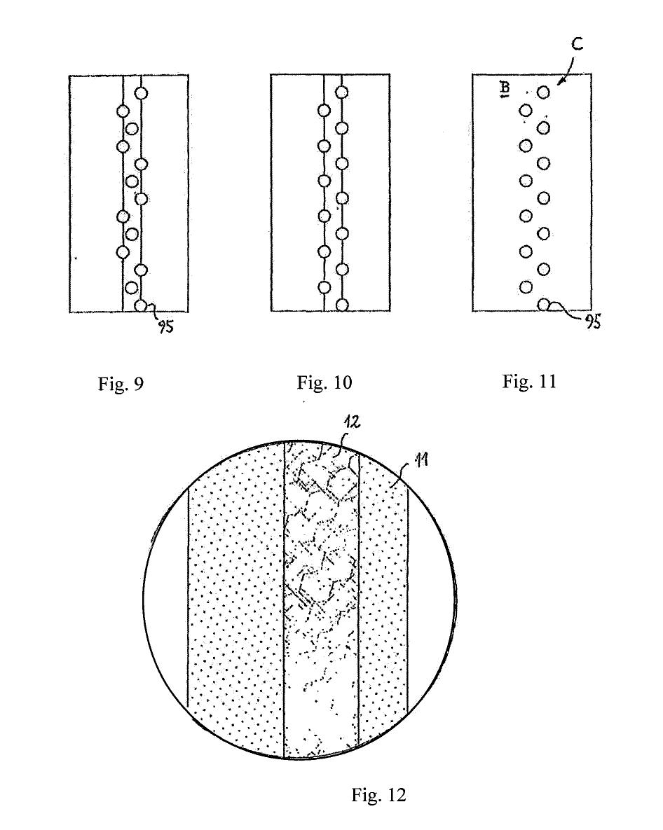

FIG. 9 shows said preform 4 having at least three layers 1, 2, 3, with a layer made of e.g. PETG being an intermediate layer 2 between the two other layers 1, 3, wherein said layer is a barrier layer, preferably a gas barrier layer, more preferably an oxygen barrier layer. In a specific embodiment, said living organisms in the PETG belong to the species Bacillus Subtilis.

Said living organisms are introduced in the PETG in a liquid stage during injection moulding, at a temperature above 200° C.

Said polymer may contain at least two different types of living organisms.

In the container made from said preform, said living organisms are active in a temperature range reaching at least 4 to 30° C. once the container 9 has been filled. At least two different types of living organisms are selected for coating granules.

Said cells are incorporated in formed vesicles or liposomes that are selected from a category of prokaryotic or eukaryotic vegetative cells and/or a phase of inactive or dormant stage, such as sexual or asexual spores, or cysts or meristematic clumps.

Said cells, mersistematic clumps or cell dormant stages, are encapsulated in the multidimensional structure, are selected to withstand extremely dry conditions and temperatures well above 100° C. and act as permanent oxygen or CO₂ barriers, UV blockers and/or potentially colorings.

The tertiary material C consists in a thermally sensitive material is added cold through a thermally unloaded way 69, in particular at substantially normal conditions of room temperature and atmosphere.

A slow and prolonged diffusion of organic molecules is realized through said polymers into the multidimensional structure and to the encapsulated microbial cells, thus creating in the polymer a moist and fluctuating environment, activating slow metabolic microbial processes.

An active and/or passive barrier for oxygen and other gases permeation is created by enlargement of immediate space within the polymer for the selected encapsulated microbial cell. This enables its slight expansion or growth and basic nutrition, enabled by degradation or not of the selected multidimensional structure. This depends on the selected microbial cell type, its metabolic activity within the liposome included in the polymer, the composition of the selected multidimensional structure as carrier.

The bio component of the polymer multidimensional structure—bioagreggates is a type of yeast with a dry spore, in particular such as Saccharomyces cerevisiae, which is able to withstand the physicochemical conditions required for the inclusion of liposome—bio-aggregates into the polymer.

Another type of yeast is pleomorphic yeasts, in particular such as Hortaea werneckii, able to grow as yeast cells, hyphal cells, budding hyphae, spores or meristematic clumps.

Another type of cells are fungal cells molds, in particular such as Aspergillus spp. and their conidia, sexual spores, hyphal fragments, mycelial strands and dormant structures, in particular such as Chlamydial cells or sclerotia.

Algal cells may be incorporated instead of fungal cells into the multidimensional structure-polymer bioagreggates, in particular such as Haematococcus or Dunaliella salina, or their spores.

The bio component is e.g. a mixture of algal and fungal cells.

The bio component of the liposome—bioagreggates is prokaryotic cells such as a type of bacterial cell with a dry spore or endospore, like Bacillus spp. or lactic acid bacteria or an archeal cell, in particular such as halophilic Halococcus or thermophilic Aeropyrum pernix.

Said living organisms may be selected from among the extremophiles. They can also be selected from among the species Bacillus Subtilis, in particular the one bearing No. ID9698.

This invention also relates to a method for producing said polymer product whereby
a polymer that is selected in form of granules;
a multidimensional structure containing amphiphilic molecules, such as lipids, surfactants or polymers, being remarkable in that the incorporation of the multidimensional structure leads to a polymer product with an improved active and/or passive oxygen or CO₂ barrier, with an improved UV blocking activity or with potentially colouring. In addition,
granules are coated by thin layer culture with said multidimensional structures, with said living organisms or with said multidimensional structures containing said living organisms, and the polymer is moulded into said product.

In particular, the polymers are selected from among the family of the thermoplastic polymers, resp. from among the family of the polyolefins, or polyesters, the family of the polyethylenes, or polypropylenes, more particularly from polyethylene terephthalate (PET). In the latter case, the PET granules are coated with living organisms that are brought to a temperature higher than 260° C. during the injection molding process.

Said polymer may also be made e.g. of PETG, with a lower melting temperature than standard PET. In said preform 4 consisting in e.g. three layers 1, 2, 3, the intermediate layer 2 is particularly made of PETG, whereas the outer layers 1, 3 are made of PET. The injection molding is a co-injection molding where the PETG is injected colder than the layers 1, 3 on both sides of it 2.

The PETG material may be fed from granules that are coated with living organisms, and be brought to a temperature higher than 200° C. during the injection molding process.

In a further embodiment, said product may be a polymer film.

As part of the invention, the abovementioned structures, with or without included microbial cells or spores, can be used in the production of plastic materials and textile to achieve considering the following remarkable advantages: a lower permeability for hydrogen protons. In textile industry this means protections against acidic environment (lab coat, glows, . . . ); in addition, a lower and controlled permeability for O₂, CO₂: in production of plastic bottles for soft drinks, fruits juice, olive and other oil sensitive to oxidation processes, and the like; still further, a lower permeability for smaller organic molecule (water filters for removing organic compounds; yet a lower permeability for water (water proofed textile, . . . ); in addition, a protection against heat and UV-light (textile, plastic, . . . ), and coloring with biological pigments, included in the cell wall or membrane of the microbial cells. Additionally coloring prevents oxidation of products in the bottle, due to scavenging abilities of pigments.

1-63. (canceled)

64. A polymer product comprising the following components:
a polymer in form of granules;
a multidimensional structure containing amphiphilic molecules, such as lipids, surfactants or polymers, wherein
said polymer product has an active and/or passive oxygen or CO₂ barrier, with an UV blocking activity or with potentially colouring with the incorporation of the multidimensional structure,
wherein it consists of a hollow form, in particular preform for manufacturing containers, that is composed of a multilayer structure comprising at least a first double base layer, that is composed of a primary material (A) consisting of a polymer, thereby forming a double primary base layer as embedding matrix, that forms the outer surface of the preform that delimitate same, and that is further comprised of a secondary intermediate layer which is comprised in said double primary basis layer, comprising a secondary material (B), wherein said secondary material (B) consists of a support material wherein a tertiary material (C) is incorporated as initiator or resp. activator material thereby forming an inner composed barrier intermediate layer.

65. The polymer product according to claim 64, wherein the multidimensional structure is a micro-emulsion, an L3 (sponge) phase, a hexagonal or a lamellar structure such as vesicles or liposomes, and/or in that the multidimensional structure contains synthetic amphiphilic molecules such as surfactants or polymers, and/or in that the multidimensional structure contains amphiphilic molecules commonly found in cell membranes of living organisms, and/or in that the multidimensional structure contains amphiphilic molecules found in archaeal micro-organisms, and/or in that the multidimensional structure is used as a carrier for living microorganisms or spores, and/or in that the multidimensional structure is a living organism.

66. The polymer product according to claim 64, wherein said primary material (A) consists of a plastic polymer, and/or wherein said carrier material (B) consists of a polymer, in particular a plastic polymer, possibly a biopolymer, and/or wherein said tertiary activator material (C) consists of a fluid, particularly wherein said fluid is formed by a liquid, in particular a viscous liquid, more particularly an oil-based liquid, a water containing or water-bound liquid, or an acrylate, possibly on oil or water basis.

67. The polymer product according to claim 64, wherein said secondary layer consists of a so-called intermediate phase such as pastes, adhesives and further materials having a liquid phase under normal conditions of pressure and temperature, and which may possibly be converted into a solid phase, in particular by hardening, more particularly wherein at least one additional intermediate layer that is composed of a quaternary material (QM), is provided at at least one contact surface of said secondary layer with respect to the primary, respectively tertiary adjacent layer, yet more particularly wherein said secondary layer constitutes less than 5%, preferably at most 1% of the total weight and/or in that said secondary layer has a very small thickness, preferably not exceeding 0.05 mm, preferably even less than 0.01 mm.

68. The polymer product according to claim 64, wherein at least one of said materials (A, B, C, D) or layers contains a predetermined quantity of additives, particularly wherein said additives consist of dying substances and/or in that said additives have a neutralizing action on external radiations and/or substances particularly electromagnetic radiations including light and/or in that said additives have a neutralizing action on reagents which have an adverse effect on a product to be contained and/or in that said additives have a neutralizing action on waste or degradation materials originating from the preform itself.

69. The polymer product according to claim 64, wherein said barrier intermediate layer is composed of so-called polymer bio-aggregate which are composed by cells and/or cell products as activator material which is worked in said secondary polymer as carrier material,
particularly wherein said cells are composed of so-called cysts and/or belong to the phase of the inactive or sleeping stages; or wherein said cells are composed of so-called yeasts; and/or wherein said cells consist in prokaryotes, in particular bacteria's, and/or eukaryotes particularly of the type protests, fungi, and/or plants.

70. The polymer product according to claim 64, wherein said cell products are composed of metabolites, i.e. the molecules which are bio-chemically synthesised by organisms.

71. The polymer product according to claim 64, wherein said organisms are unicellular or multicellular, and/or wherein it comprises a polymer containing living organisms, particularly being introduced in the polymer in a liquid stage during injection molding, at a temperature above 260° C., more particularly which is intended for producing containers made of a polymer containing living organisms, consisting of at least one layer, wherein said layer is made of a polyethylene terephthalate glycol containing living organisms.

72. The polymer product according to claim 71, wherein said polymer product comprises a layer of a preform including at least three layers, the layer made of PETG being an intermediate layer between the two other layers,
particularly wherein said polymer product comprises a layer of a preform intended to make a container, said layer being a barrier layer, preferably a gas barrier layer, more preferably an oxygen barrier layer.
more particularly wherein said living organisms in the PETG belong to the species Bacillus Subtilis;
yet more particularly wherein said living organisms are introduced in the PETG in a liquid stage during injection moulding, at a temperature above 200° C.

73. A container made from a preform comprising a polymer product as defined in claim 64, wherein said polymer contains at least two different types of living organisms,
particularly wherein said living organisms are active in a temperature range reaching at east 4 to 30° C. once the container has been filled,
more particularly wherein at least two different types of living organisms are selected for coating granules.

74. A method for manufacturing a polymer product, according to claim 64, wherein said cells incorporated in formed vesicles or liposomes are selected from a category of prokaryotic or eukaryotic vegetative cells and/or a phase of inactive or dormant stage, such as sexual or asexual spores, or cysts or meristematic clumps, particularly wherein said cells, mersistematic clumps or cell dormant stages, which are encapsulated in the multidimensional structure, are selected to withstand extremely dry conditions and temperatures well above 100° C. and act as permanent oxygen or CO₂ barriers, UV blockers and/or potentially colorings.

75. The method according to claim 74, wherein the tertiary material (C) consisting in a thermally sensitive material is added cold by means of a thermally non loaded way, in particular at substantially normal conditions of room temperature and atmosphere.

76. The method according to claim 74, wherein a slow and prolonged diffusion of organic molecules is realized through said polymers into the multidimensional structure and to the encapsulated microbial cells, thereby creating in the polymer a moist and fluctuating environment, that activates slow metabolic microbial processes.

77. The method according to claim 74, wherein an active and/or passive barrier for oxygen and other gases permeation is created by enlargement of immediate space within the polymer for the selected encapsulated microbial cell, enabling its slight expansion or growth and basic nutrition, enabled by degradation or not of the selected multidimensional structure, depending on the selected microbial cell type, its metabolic activity within the liposome included in the polymer, the composition of the selected multidimensional structure as carrier.

78. The method according to claim 74, wherein the bio component of the polymer multidimensional structure—bioagreggates is a type of yeast with a dry spore, in particular such as Saccharomyces cerevisiae, which is able to withstand the physicochemical conditions required for the inclusion of liposome—bio-aggregates into the polymer;
particularly wherein another type of yeast is pleomorphic yeasts, in particular such as Hortaea werneckii, able to grow as yeast cells, hyphal cells, budding hyphae, spores or meristematic clumps.

79. The method according to claim 74, wherein another type of cells are fungal cells molds, in particular such as Aspergillus spp. and their conidia, sexual spores, hyphal fragments, mycelial strands and dormant structures, in particular such as Chlamydial cells or sclerotia;
particularly wherein instead of fungal cells, algal cells are incorporated into the multidimensional structure—polymer bioagreggates, in particular such as Haematococcus or Dunaliella salina, or their spores.

80. The method according to claim 74, wherein the bio component is a mixture of algal and fungal cells, and/or
wherein the bio component of the liposome—bioagreggates is prokaryotic cells such as a type of bacterial cell with a dry spore or endospore, like Bacillus spp. or lactic acid bacteria or an archeal cell, in particular such as halophilic Halococcus or thermophilic Aeropyrum pernix.

81. The method according to claim 74, wherein said living organisms are selected from among the extremophiles;
particularly wherein said living organisms are selected from among the species Bacillus Subtilis, in particular the one bearing No. ID9698.

82. The method for producing the polymer product according to claim 74, wherein said product comprises the following components:
a polymer that is selected in form of granules;
a multidimensional structure containing amphiphilic molecules, such as lipids, surfactants or polymers, characterized in that
the incorporation of the multidimensional structure leads to a polymer product with an improved active and/or passive oxygen or CO₂ barrier, with an improved UV blocking activity or with potentially colouring,
granules are coated by thin layer culture with said multidimensional structures, with said living organisms or with said multidimensional structures containing said living organisms,
the polymer is moulded into said product;
particularly wherein the polymers are selected from among the family of the thermoplastic polymers;
more particularly wherein the polymers are selected from among the family of the polyolefins, or polyesters;
yet more particularly wherein the polymers are selected from among the family of the polyethylenes, or polypropylenes;
still more particularly wherein said polymer is selected from polyethylene terephthalate (PET); even more particularly wherein the PET granules coated with living organisms are brought to a temperature higher than 260° C. during the injection molding process; or wherein said polymer is made of PETG, with a lower melting temperature than standard PET.

83. The method according to claim 74, wherein said product is a preform intended to be further processed to a container, particularly by injection blow moulding,
particularly wherein said preform consists in at least three layers, in particular an intermediate layer, more particularly made of PETG, between two outer layers;
yet more particularly wherein said outer layers are made of PET;
still more particularly wherein the injection molding is a co-injection molding where the PETG is injected colder than the layers on both sides of it; even more particularly wherein the PETG material is fed from granules that are coated with living organisms, and in that it is brought to a temperature higher than 200° C. during the injection molding process.

84. The method according to claim 74, characterized in that work is carried out at the working temperature range taken from the temperature interval of which the lower limit is set at substantially 100° C. under substantially standard pressure conditions, in particular at substantially one atmosphere, preferably at a temperature of 30° to 40° C.

85. The method according to claim 74, wherein said barrier materials (B & C) are applied uniformly with very low quantities material, particularly tertiary material (C).

86. The method according to claim 74, characterized in that said product is worked as a polymer film.
